(19) United States
(12) Patent Application Publication
(10) Pub. No.: US 2005/0058676 A1
(43) Pub. Date: Mar. 17, 2005

(54) Composition comprising at least one conductive polymer and at least one rigid non-film-forming particle, and processes for the uses thereof

(76) Inventors: Isabelle Rollat-Corvol, Paris (FR); Henri Samain, Bievres (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/892,334

(22) Filed: Jul. 16, 2004

Related U.S. Application Data
(60) Provisional application No. 60/492,298, filed on Aug. 5, 2003.

(30) Foreign Application Priority Data
Jul. 16, 2003 (FR) 03 08678

(51) Int. Cl. A61K 7/06; A61K 7/11

(52) U.S. Cl. 424/401; 424/70.17

(57) ABSTRACT

The disclosure relates to a composition comprising, in a cosmetically acceptable medium, (a) at least one rigid non-film-forming particle and (b) at least one conductive polymer. The disclosure also relates to a process for treating keratin fibers, a process for shaping and/or holding the hairstyle, and a process for giving keratin fibers an optical effect.

This application claims benefit of U.S. Provisional Application No. 60/492,298, filed Aug. 5, 2003.

The present disclosure relates to a composition comprising, in a cosmetically acceptable medium, at least one conductive polymer and at least one rigid non-film-forming particle. The disclosure also relates to a process for treating keratin fibers using the above mentioned composition and also a process for shaping and/or holding the hairstyle comprising applying a hair product base to the hair, the hair product base comprising the composition disclosed herein. Finally, the present disclosure relates to a process for imparting to the keratin fibers an optical effect.

The present invention relates to the field of treating keratin fibers, for example human keratin fibers such as the hair.

Most of the treatments applied to keratin fibers, such as dyeing, bleaching or permanent-reshaping processes, may have major consequences on the characteristics of the fibers, and especially on their sheen. Thus, following repeated treatments, it is not uncommon to find that the treated fibers become more or less dull, despite the improvements that have been made in the processes used.

In order to compensate for these negative effects, and with the aim of giving the hair sheen, it is known practice to use, for example, lubricating hydrophobic substances such as organic oils or waxes or silicones.

However, the sheen effect obtained may lack intensity and generally gives the fibers an artificial appearance.

In addition, these compositions may give the fibers an undesirable greasy or tacky feel.

One aim of the compositions disclosed herein is to give treated keratin fibers an optical aspect, for instance sheen, without any or without at least one of the drawbacks encountered with standard compositions.

Moreover, in certain cases, the compositions disclosed herein may give color to the keratin fibers to which they are applied.

Thus, disclosed herein is a composition comprising, in a cosmetically acceptable medium:
(a) at least one rigid non-film-forming particle, and
(b) at least one conductive polymer.

The invention moreover relates to a process for treating keratin fibers, for example, human keratin fibers such as the hair, wherein the composition disclosed herein is applied to the wet or dry fibers, and the fibers are then dried or are left to dry.

Also disclosed herein is a process for styling and/or fixing the hair comprising applying a hair product base to the hair, the hair product base comprising the composition disclosed herein.

Another embodiment of the disclosure is a process for imparting to keratin fibers an optical effect comprising applying to the keratin fibers a composition comprising at least one rigid non-film-forming-particle and at least one conductive polymer.

Specifically, the composition disclosed herein may give the fibers a sheen that is more intense, more natural and/or more aesthetic than with the means of the prior art.

Moreover, when the conductive polymers present in the composition disclosed herein absorb light in the visible spectrum, an optical effect, for instance a sheen, and color are obtained simultaneously.

Finally, fibers treated with the composition disclosed herein may have a soft, non-greasy feel.

However, other characteristics and advantages of the invention will emerge more clearly on reading the description and the examples that follow.

In the text herein below and unless otherwise indicated, the limits of a range of values are understood as forming part of that range.

For the purposes of the present invention, the term “optical effect” covers sheen, color, metallic, goniochromatic, and moiré effects.

Moreover, it should be noted that the sheen corresponds to the light intensity reflected at an angle α when the lock of hair is illuminated under an angle α. The angle α conventionally used to measure this specular reflection, in other words the sheen, is equal to 20°. This provision of sheen may be measured using a glossmeter as described, for example, in ISO standard 2813-1994 from AFNOR (August 1994, amended February 1997).

Conductive Polymers

According to the present invention, the term “conductive polymer” means a molecular structure in which the monomer(s) has (have) high electron delocalization and whose arrangement in the polymer skeleton allows the π orbitals to overlap. This chemical characteristic is reflected by electrical conduction, which may or may not be accompanied by absorption in the UV-visible spectrum, or even in the infrared spectrum.

For the purposes of the present disclosure, the expression “conductive polymer absorbing in the visible spectrum” means any conductive polymer having a non-zero absorbance in the wavelength ranging from 400 to 800 nm, even if the absorption maxima of the polymer are outside this range.

The conductive polymers used in the context of the present disclosure are conductive polymers that are soluble or dispersible in the cosmetic medium suitable for use.

The polymer is said to be soluble in the medium when it forms an isotropic clear liquid at 25° C. in the medium comprising water or a water/solvent mixture, this being obtained throughout all or part of a concentration ranging from 0.01% to 50% by weight of conductive polymer.

The conductive polymers used in the context of the present disclosure may be conductive polymers that are soluble or dispersible in an aqueous medium such as in water.

The polymer is said to be dispersible in the medium comprising water or a water/solvent mixture if, at 0.01% by weight, at 25° C., it forms a stable suspension of fine, generally spherical particles. The mean size of the particles constituting the said dispersion is less than 1 μm, more generally ranging from 5 to 400 nm, for example, from 10 to 250 nm. These particle sizes are measured by light scattering.

It should be noted that these polymers may not require the use of a dispersant.

The conductive polymers may be, for example, in a form that is soluble in the medium of the composition.

Furthermore, the polymers may, for example, have a conductivity ranging from 10⁻⁵ and 5×10⁵ siemens/cm, for example, from 10⁻³ to 10⁵ siemens/cm such as from 10⁻¹ to 10⁴ siemens/cm.

The conductivity is measured using a current generator (RM2 Test Unit sold by the company Jandel) equipped with a four-point measuring head (Universal four-point probes sold by the company Jandel). The four points, aligned and separated by the same space d, are applied by simple pressure to the sample to be analyzed. A current I is injected via the outer points using the current source, thus creating a variation in potential. The voltage U is measured between the two inner points connected to the voltmeter of the current generator.

In this configuration, the conductivity of the sample expressed in S/cm is given by the following expression:
σ=(K×I)/(U×e)
wherein:
K is a coefficient depending on the position of the contacts on the surface of the sample. When the points are aligned and equidistant, K is equal to: π/log(2)
I: value of the injected current, expressed in amperes
U: the measured voltage value, expressed in volts
e: thickness of the sample, expressed in cm.

This expression can be used only when the thickness of the material is negligible compared with the distance d existing between two points (e/d<0.25). In order to obtain sufficiently small thicknesses and thus to be able to calculate the conductivity of the material, one may perform the measurement on a non-conductive support (for example a glass slide) coated with the material to be analyzed, obtained by evaporation of a dilute solution. In order to improve the homogeneity of the coating to be analyzed, one may use the deposition technique known as spin coating.

According to one non-limiting embodiment, the conductive polymers present in the compositions disclosed herein are chosen from polymers comprising at least one repeating unit of the following formulae:
anilines of structure (I) below: 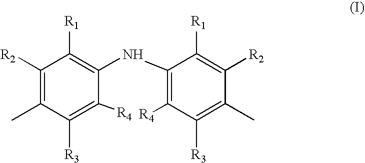
pyrroles of structures (IIa) and (IIB) below: 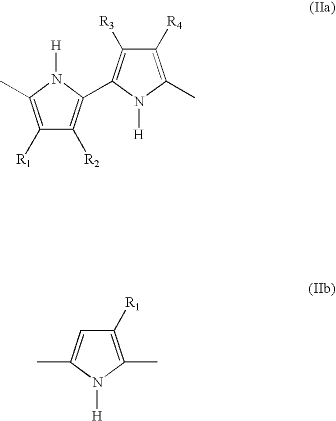
   thiophenes or bisthiophenes of formulae (IIIa), (IIIb) and (IIIc) below: 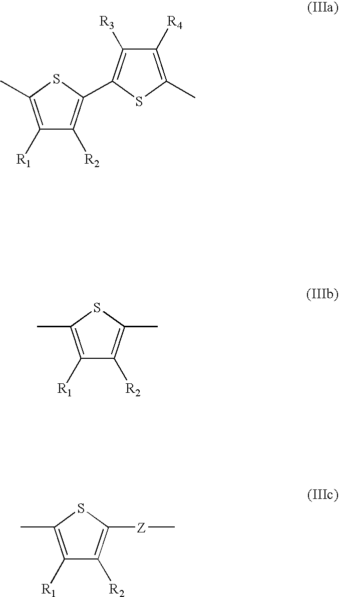
furans of formula (IV) below: 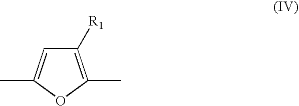
para-phenylene sulfides of structure (V) below: 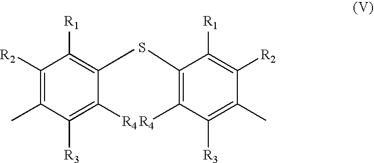
para-phenylenevinylenes of formula (VI) below: 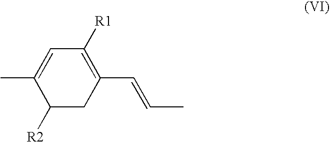
indoles of formula (VII) below: 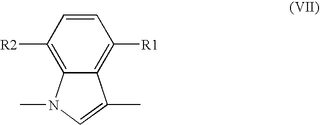
aromatic amides of formulae (VIIIa), (VIIIb), (VIIIc) and (VIIId) below: 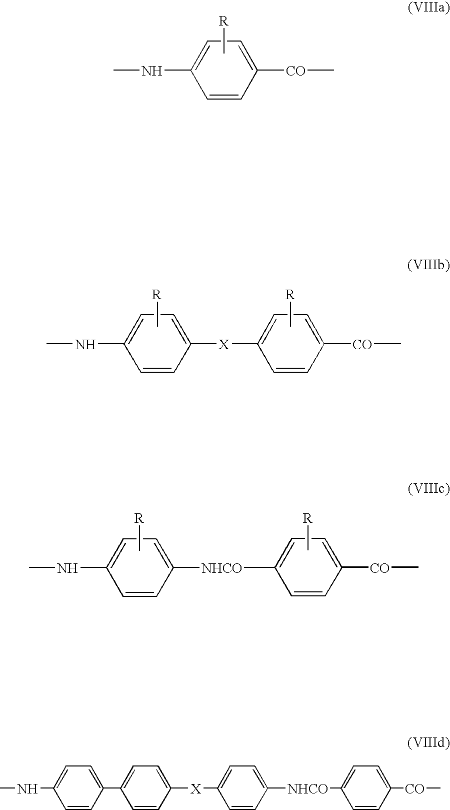
aromatic hydrazides of formulae (IXa), (IXb) and (IXc) below: 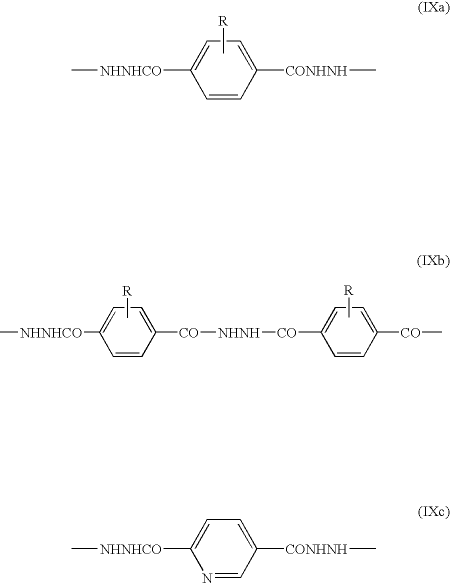
the aromatic azomethines of formulae (Xa), (Xb) and (Xc) below: 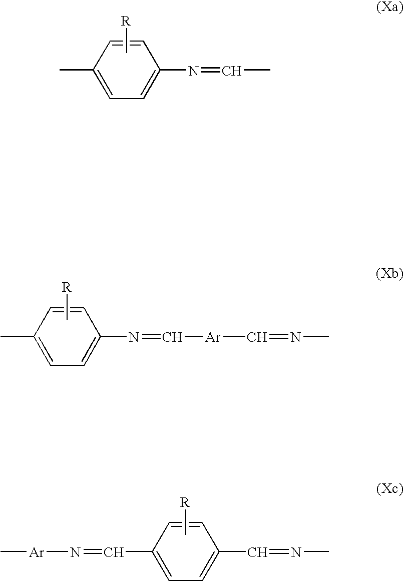
aromatic esters of formulae (XIa), (XIb) and (XIc) below: 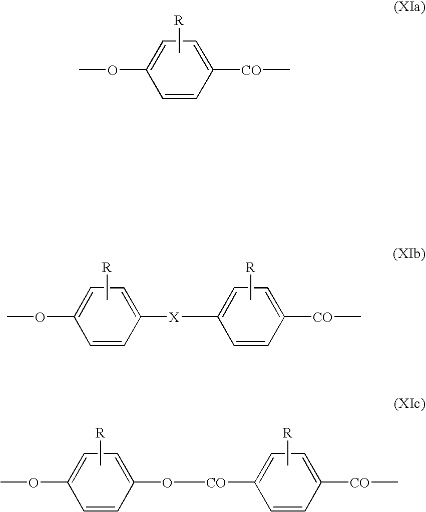
wherein in formulae (I) to (XI):
the radicals R, R₁, R₂, R₃, and R₄, which may be identical or different, are chosen from hydrogen, —R′ radicals, —OR′ radicals, —COOR′ radicals, and —OCOR′ radicals, wherein R′ is chosen from linear and branched C₁-C₂₀ alkyl radicals, halogen atoms, nitro radicals, cyano radicals, cyanoalkyl radicals, and solubilizing groups optionally comprising a spacer group that bonds to the ring;
Ar is a radical comprising a monoaromatic or a polyaromatic radical;
X is a radical chosen from —NHCO—, —O—, —S—, —SO₂—, —N═N—, —C(CH₃)₂—, —CH₂—, —CH═CH—, and —CH═N— radicals; and
Z is a radical chosen from —CH═CH— and —C≡C— radicals.

For example, Ar may be at least one radical chosen from the following:

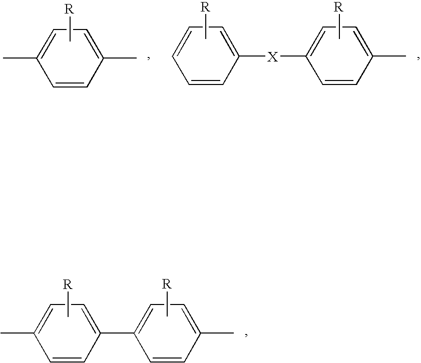

For the purposes of the present disclosure, the term “solubilizing group” means a group that ensures the dissolution of the said molecule in the cosmetic medium, such that the polymer has a conductive nature after drying the composition.

The at least one conductive polymer present in the composition disclosed herein may comprise at least one repeating unit comprising at least one solubilizing group, and at least one other such unit lacking such groups.

The solubilizing groups may, for example, be chosen from:
carboxylic (—COOH) and carboxylate (—COO-M+) radicals, wherein M is chosen from alkali metals, such as sodium or potassium, alkaline-earth metals, organic amines such as a primary, secondary or tertiary amine, alkanolamines and amino acids,
sulfonic (—SO₃H) and sulfonate (—SO₃⁻M⁺) radicals, M having the same definition as above,
primary, secondary and tertiary amine radicals,
quaternary ammonium radicals such as —N(R′)₃⁺Z⁻ wherein Z is chosen from Br, Cl, (C₁-C₄)alkyl-OSO₃ radicals; and R′, which may be identical or different, is chosen from linear and branched C₁ to C₂₀ alkyl radicals, or two R's form a heterocycle with the nitrogen,
hydroxyl radicals, and
poly((C₂-C₃)alkylene oxide) radicals.

The carboxylic or sulfonic acid functions may or may not be neutralized with a base, such as sodium hydroxide, 2-amino-2-methylpropanol, triethylamine or tributylamine, for example.

The amine radicals may or may not be neutralized with a mineral acid, such as hydrochloric acid, or with an organic acid, such as acetic acid or lactic acid, for example.

In addition, the solubilizing groups may be connected to the ring via a spacer group, for instance a radical chosen from —R″—, —OR″—, —OCOR″— and —COOR″— wherein R″ is chosen from linear and branched C₁-C₂₀ alkyl radicals optionally comprising at least one hetero atom, for instance oxygen.

For example, the radicals R, R₁, R₂, R₃ and R₄, which may be identical or different, are chosen from hydrogen, R′, —OR′, —OCOR′, and —COOR′ radicals, wherein R′ is chosen from linear and branched C₁-C₆ alkyl radicals, and from the following neutralized or non-neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, —[(CH₂)₂O]ₓCH₂CH₂OCH₃ with x being an average number ranging from 0 to 200.

The number n of repeating units in the polymer usually ranges from 5 to 10 000, for example, from 5 to 1000, for further example from 10 to 1000 such as from 20 to 700.

The conductive polymer may be one, for example, such that at least one radical chosen from R, R₁, R₂, R₃, and R₄ is a solubilizing group.

In accordance with one embodiment of the disclosure, the at least one conductive polymer used comprises at least one solubilizing group per repeating unit. Thus, for example, at least one radical chosen from R, R₁, R₂, R₃, and R₄ is a solubilizing group.

The conductive polymer may be, for example, soluble in the medium of the composition.

The at least one conductive polymer present in the composition disclosed herein is well known to those skilled in the art and is described in the book “Handbook of Organic Conductive Molecules and Polymers” Vol. 1-3 (Wiley 1997), and also in the review Can. J. Chem. Vol. 64 (1986).

Polythiophenes and their synthesis are described in the article taken from the review Chem. Mater. 1998, Vol. 10, No 7, pages 1990-1999 by the authors Rasmussen, S. C.; Pickens, J. C.; and Hutchison, J. E. “A new, general approach to tuning the properties of functionalized polythiophenes: The oxidative polymerization of monosubstituted bithiophenes”; the article taken from the review Macromolecules 1998, 31, pages 933-936, by the same authors “Highly conjugated, water-soluble polymers via direct oxidative polymerization of monosubstituted bithiophenes.” In addition to polymerization via chemical or electrochemical oxidation, they may also be obtained by polycondensation (dihalothiophene; catalysis with nickel or palladium complexes); via Suzuki coupling (coupling between a halogen function, for example bromine, and a boronic acid, catalysis: palladium complex and base; this then gives coupling of AA-BB type (reaction of monomers of the type A-X-A with B-X′-B) or of A-B type (reaction of several monomers of the type A-X-B); via Stille coupling (formation of a carbon-carbon bond in the presence of a Pd-based catalyst—AA-BB or A-B type); via Reike polymerization (organozinc in the presence of a nickel complex); via polymerization of McCulloch type, etc.

The at least one conductive polymer present in the composition disclosed herein is also described in international patent application WO 99/47570.

Among the conductive polymers suitable for the composition disclosed herein, mention may be made of the polymers comprising at least one repeating unit of formulae (IIIa), (IIIb) or (IIIc) wherein the solubilizing groups may, for example, be chosen from carboxylic acid groups; sulfonic acid groups; tertiary amine radicals; quaternary ammonium radicals such as —N(R′)₃⁺Z⁻ radicals wherein Z is chosen from Br, Cl, and (C₁-C₄)alkyl-OSO₃ radicals. and R′, which may be identical or different, is chosen from linear and branched C₁ to C₂₀ alkyl radicals, or two R's form a heterocycle with the nitrogen; the groups being optionally connected to the ring via a spacer group. The carboxylic or sulfonic acid functions may optionally be neutralized.

Thus, the polymerization may be performed via chemical or electrochemical oxidation of the corresponding thiophene monomer or else via polycondensation.

By way of illustration, the polythiophenes of formulae (IIIa) and (IIIb) may be obtained by polymerization via oxidation (for example with FeCl₃ catalysis); via polycondensation of dihalothiophene catalysed with nickel or palladium complexes (e.g.: NiCl₂(dppe)₂); via Suzuki coupling (coupling between a halogen function, for example bromine, and a boronic acid, catalysis: palladium complex and base; this then gives coupling of AA-BB type (reaction of monomers of the type A-X-A with B-X′-B) or of A-B type (reaction of several monomers of the type A-X-B); via Stille coupling (formation of a carbon-carbon bond formed in the presence of a Pd-based catalyst—M-BB or A-B type); via Reike polymerization (organozinc in the presence of a nickel complex); via polymerization of McCulloch type, etc.

The vinylene polythiophenes of formula (IIIc) wherein Z is a —CH═CH— radical may be obtained, for example, via Gilch polymerization in the presence of a strong base (potassium tert-butoxide) of 2,5-bis(bromoalkylene)thiophene; via polymerization by the Wessling method via the use of a precursor based on sulfonium salts and pyrolysis; via a Wiftig-Horner Wittig reaction.

The ethynylene polythiophenes of formula (IlIc) wherein Z is a —C≡C— radical may be obtained by Heck-Sonogashira coupling (of the type AA-BB or A-B; formation of a carbon-carbon bond between a terminal acetylenic (or true acetylenic) function and a bromo or iodo function, catalysed with a palladium/copper complex (PdCl₂(PPh₃)₃, CuI or Cu(OAc)₂) in the presence of a base such as triethylamine, diisopropyl amine, piperidine, etc.); via metathesis of alkynes in the presence of a molybdenum complex (Mo(CO)₆).

In general, the functionalization of the polythiophenes, in other words the introduction of the solubilizing or non-solubilizing group(s), is performed on the monomer before it is polymerized.

In certain cases, the solubilizing group is obtained after working up the polymer. For example, this is the case for the carboxylic acid function, which may be obtained by hydrolysis of the corresponding ester.

For example, the solubilizing groups are chosen from carboxylic acid groups; sulfonic acid groups; tertiary amine radicals; quaternary ammonium radicals such as —N(R′)₃⁺Z⁻ radicals wherein Z is chosen from Br, Cl, and (C₁-C₄)alkyl-OSO₃ radicals, and R′, which may be identical or different, is chosen from linear and branched C₁-C₂₀ alkyl radicals, optionally connected to the ring via a spacer group, such as a C₁-C₂₀ alkyl radical; and also the salts thereof. The carboxylic or sulfonic acid functions may optionally be neutralized.

According to one embodiment of the invention, the at least one conductive polymer comprises at least one repeating unit of formula (IIIa), (IIIb) or (IIIc), wherein at least one radical chosen from R₁, R₂, R₃, and R₄ of formula (IIIa) or R₁ and R₂ of formulae (IIIb) or (IIIc) is a carboxylic acid type solubilizing group, in neutralized or non-neutralized form, optionally connected to the ring via a spacer group such as a linear or branched C₁-C₂₀ alkyl radical, the other radical(s) being hydrogen atoms.

The at least one conductive polymer is generally present in the composition in an amount of at least 0.001% by weight, for example, at least 0.01% by weight, for further example at least 0.1% by weight such as at least 0.5% by weight, relative to the total weight of the composition. Moreover, the amount of conductive polymer present may be up to 50% by weight, for example, up to 30% by weight, such as up to 20% by weight and for further example up to 10% by weight, relative to the total weight of the composition.

According to one embodiment of the invention, the conductive polymer is present in an amount ranging from 0.1% to 50% by weight, for example from 0.1% to 30% by weight such as from 0.5% to 10% by weight, relative to the total weight of the composition.

As indicated previously, the composition disclosed herein comprises at least one rigid non-film-forming particle.

The particle may be of organic or mineral nature.

In general, the particle may be spherical or non-spherical. In the second case, they may be ovoid, in the form of platelets or in the form of tubes (irrespective of the shape of the cross section, circular or non-circular). They may also be polygonal, for instance, of cubic, parallelepipedal, tetrahedral, etc. shape.

Their mean size may range from 1 nm to 5 μm in the longest length, in the case of spheres, ovoids or platelets. For example, the mean particle size is less than or equal to 1 μm, such as less than or equal to 500 nm and, for further example, less than or equal to 300 nm.

In the case where the particles are in the form of a tube or other forms different from those first mentioned, their mean size usually ranges from 10 nm to 100 μm.

According to one embodiment, the rigid non-film-forming particles are of mineral nature and may be chosen, inter alia, from oxides and silicates, such as alkali metals or of alkaline-earth metals; silica; titanium dioxide; alumina; aluminosilicates; calcium carbide, silicon nitrite and native metals, for instance gold, and alloys thereof.

According to another embodiment, the rigid non-film-forming particles are of organic nature and may be, for example, polymeric.

In another embodiment, the rigid non-film-forming particles are polymer particles with a glass transition temperature of greater than 50° C., for example, greater than 70° C.

The size polydispersity of the polymer particles dispersed in the medium of the compositions of the invention, measured by quasi-elastic light scattering, may be, for example, less than 0.35.

The at least one rigid non-film-forming polymer particle of the composition disclosed herein may comprise a crosslinked polymer.

The crosslinking agents may be chosen, for example, from those commonly used in free-radical polymerization. Examples that may be mentioned include the diacrylates or dimethacrylates of ethylene glycol, of polyethylene glycol, of propylene glycol or of divinylbenzene, and pentaerythrityl di- or trimethacrylate; diacrylates or dimethacrylates of alkylenediols, for instance hexanediol dimethacrylate. They are used in amounts ranging from, for example, 0.1% to 50% by weight relative to the weight of the monomers constituting the polymer of the latex.

In aqueous media containing volatile organic compounds, for example, those present in the aerosol lacquers or pump-dispenser bottles of the disclosure, a dispersion of polymer particles comprising ionized or ionizable anionic groups, such as carboxylic acid or sulfonic acid groups, may be used, for example, to give the latex good stabilization (in particular in an aqueous-alcoholic medium).

These acidic groups may be present, for example, in amounts of less than or equal to 10% by weight, such as less than or equal to 8% by weight and for further example ranging from 3% to 8% by weight relative to the weight of the polymer.

These acidic groups may be, for example, partially or totally neutralized with a volatile mineral base or an amino alcohol as defined above.

Among the polymers constituting the rigid non-film-forming particles, examples that may be mentioned include polymers or copolymers, which may be, for example, crosslinked, obtained by polymerization or copolymerization of a monomer or a blend of monomers chosen from linear, cyclic and branched C₁-C₁₀ alkyl acrylates and methacrylates, such as methyl methylmethacrylate, tert-butyl methacrylate, cyclohexyl methacrylate and isobornyl acrylate and methacrylate; styrene; vinyltoluene; vinyl chloride, vinyl benzoate and vinyl tert-butylbenzoate; acrylic acid, and methacrylic acid.

According to one embodiment of the invention, the rigid non-film-forming particles are cationic latices. The term “cationic latices” means particles whose surface charge is positive (partially or more).

In accordance with another embodiment, the particles are formed from conductive polymers.

The composition disclosed herein may also comprise at least one surfactant, which may be chosen from anionic, amphoteric, nonionic, zwitterionic and cationic surfactants, and mixtures thereof.

Among these surfactants, mention may be made of alkyl sulfates, alkylbenzene sulfates, alkyl ether sulfates, alkyl sulfonates, quaternary ammonium salts, alkylbetaines, oxyethylenated alkylphenols, fatty acid alkanolamides, oxyethylenated fatty acid esters and other nonionic surfactants of the hydroxypropyl ether type.

When the composition comprises one or more surfactants, their content is usually less than 30% by weight, for example, from 0.5% to 10% by weight relative to the weight of the composition.

The composition may similarly comprise at least one non-fluorescent direct dye.

The direct dye may be of nonionic, cationic or anionic nature.

Generally, these direct dyes are chosen from nitrobenzene dyes, azo, azomethine, methine, anthraquinone, naphthoquinone, benzoquinone, phenothiazine, indigoid, xanthene, phenanthridine, phthalocyanin and triarylmethane-based dyes, or natural dyes (for instance henna or camomile), and mixtures thereof.

When present, the at least one direct dye is present in an amount ranging from 0.0005% to 12% by weight relative to the total weight of the composition, such as from 0.005% to 6% by weight relative to the total weight of the composition.

The composition may further comprise at least one fluorescent dye and/or at least one optical brightener.

It should be noted that fluorescent dyes are, more specifically, compounds chosen from those that absorb light in the visible part of the spectrum and possibly in the ultraviolet region, and re-emit fluorescent light in the visible part of the spectrum, of a longer wavelength than that of the absorbed light. The wavelength of the light re-emitted by the fluorescent compound ranges, appropriately, from 500 to 650 nm.

Optical brighteners are compounds chosen from those that absorb light in the ultraviolet part of the spectrum, essentially in the UVA range, at a wavelength ranging from 300 to 390 nm. These compounds re-emit fluorescent light in the visible part of the spectrum, between 400 and 525 nm.

Fluorescent dyes and/or optical brighteners that are soluble in the medium of the composition, at room temperature (from 15 to 25° C.), may, for example, be used. For example, the solubility of the fluorescent dye or of the optical brightener in the medium of the composition is at least 0.001 g/l and such as at least 0.5 g/l, at a temperature ranging from 15 to 25° C.

The fluorescent dyes that are suitable for the disclosure may be chosen from, for example, substituted 4-aminophenylethenylpyridinium derivatives; naphthalimides; cationic and non-cationic coumarins; xanthenodiquinolizines; azaxanthenes; naphtholactams; azlactones; oxazines; thiazines; dioxazines, pyrenes and nitrobenzoxadiazoles, and mixtures thereof.

In addition, if they are present, the amount of fluorescent dye and optical brightener may each range, for example, from 0.01% to 20% by weight, for further example, from 0.05% to 10% by weight such as from 0.1% to 5% by weight relative to the total weight of the composition.

Moreover, the cosmetically acceptable medium of the composition is water or a mixture of water and at least one organic solvents that are acceptable in the field.

Among the solvents that may be suitable are C2-C4 aliphatic monoalcohols, such as ethyl alcohol or isopropyl alcohol, aromatic alcohols, for instance benzyl alcohol and phenylethyl alcohol, glycols or glycol ethers, for instance ethylene glycol monomethyl ether, monoethyl ether and monobutyl ether, propylene glycol or ethers thereof, for instance propylene glycol monomethyl ether, butylene glycol, dipropylene glycol and diethylene glycol alkyl ethers, for instance diethylene glycol monoethyl ether or monobutyl ether, or alternatively polyols such as glycerol.

The composition may also comprise an effective amount of other agents, which are previously known elsewhere in the treatment of human keratin fibers, such as fixing polymers, thickeners, film-forming polymers, styling polymers, antioxidants, fragrances, dispersants, conditioners especially including cationic or amophoteric polymers, opacifiers, UV-screening agents, preserving agents, ceramides, pseudoceramides, vitamins or provitamins, for instance panthenol, and nonionic, anionic, amphoteric or cationic associative polymers.

The composition according to the invention may be in the form of a lotion, a spray, a mousse, a cream or a gel or in any other suitable form.

For example, the composition as disclosed herein may be packaged under pressure in a pump-dispenser bottle or in a pressurized aerosol device.

In the case of the aerosol device, the composition comprises at least one propellant that may be chosen from volatile C3-C5 hydrocarbons, such as n-butane, propane, isobutane and pentane; chloro and/or fluoro hydrocarbons and mixtures thereof; carbon dioxide, nitrous oxide, dimethyl ether, nitrogen and compressed air, and mixtures thereof.

The concentration of propellent gas in the aerosol device depends on the nature of the chosen propellant.

By way of illustration, the amount of propellant ranges from 5% to 90% by weight relative to the total weight of the composition in the aerosol device, for example, ranging from 10% to 60% by weight relative to the total weight of the composition in the aerosol device.

The composition that has just been described is generally applied to wet or dry keratin fibers, and the fibers are then dried or left to dry.

In one embodiment, the fibers are shaped at the time of drying.

This drying operation generally takes place within a temperature range ranging from 20 to 120° C. such as from 20 to 80° C.

The example that follows illustrates the invention without, however, being limiting in nature.

### EXAMPLE

Synthesis of poly(thiophene-3-acetic Acid)

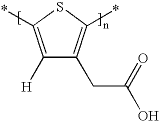

Procedure

Preparation of the Polymer: poly(ethyl thiophene-3-acetate)

25 ml of dry chloroform were introduced into a Schlenk tube under argon, the system was degassed and the following reagents were then introduced: 2.5 g of ethyl thiophene-3-acetate (14.7 mmol) 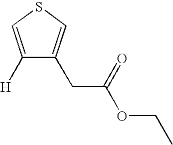
and 1 g of FeCl₃ (6.15 mmol).

The mixture was stirred for 24 hours under argon at 50° C.

The poly(ethyl thiophene-3-acetate) polymer was then precipitated in heptane.

The polymer was then dissolved in a tetrahydrofuran solution.

Infrared characterization:

C═O band: 1719 cm⁻¹; CH₂, CH₃ bands=2979 cm⁻¹, 2934 cm⁻¹ and disappearance of the CH band at 3102 cm⁻¹ present in the monomer.

Hydrolysis of the polymer: poly(ethyl thiophene-3-acetate) to form poly(thiophene-3-acetic acid)

The polymer obtained above was then hydrolysed with an excess of 50 ml of an aqueous sodium hydroxide solution (2N) for 48 hours at 70° C., followed by acidification with concentrated HCl up to the point of precipitation of the product: poly(thiophene-3-acetic acid).

The polymer was then filtered off and washed several times with distilled water in order to remove the traces of catalyst.

Infrared Characterization of the Polymer:

C═O band: 1740 cm⁻¹; COO 1580 cm⁻¹; OH (broad band 3000-3500 cm⁻¹)

Neutralization of the poly(thiophene-3-acetic acid) polymer:

The poly(thiophene-3-acetic acid) polymer (2 g) was dissolved in tetrahydrofuran (30 g) and neutralized with a proportion of 1 mol of sodium hydroxide per mole of carboxylic acid.

Water (30 g) was then added.

The tetrahydrofuran was evaporated off.

An aqueous 6% solution of poly(thiophene-3-acetic acid) in the form of a sodium salt was thus obtained.

Formulation comprising the polymer and process using it:

| | |
|---|---|
| Poly(thiophene-3-acetic acid) | 4 g |
| Aminomethyl propanol qs | pH 7 |
| Basoplast 265D (from BASF) | 1 g |
| Ethyl alcohol | 15 g |
| Water qs | 100 g |

The formula was applied to dark hair. After a standing time of 5 minutes, drying (free drying) was performed.

1. A composition comprising, in a cosmetically acceptable medium:
(a) at least one rigid non-film-forming particle, and
(b) at least one conductive polymer.

2. A composition according to claim 1, wherein the at least one conductive polymer comprises at least one repeating unit chosen from the following formulae:
anilines of structure (I) below: 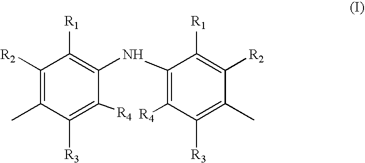
pyrroles of structures (IIa) and (IIb) below: 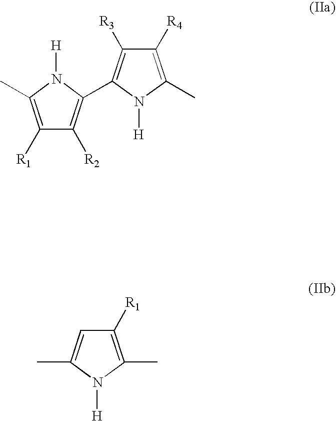
thiophenes or bisthiophenes of formulae (IIIa), (IIIb) and (IIIc) below: 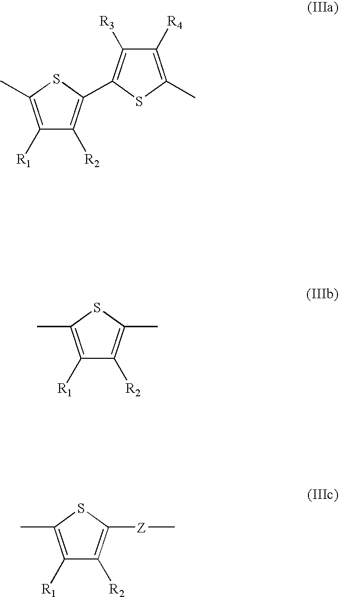
furans of formula (IV) below: 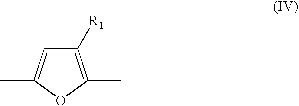
para-phenylene sulfides of structure (V) below: 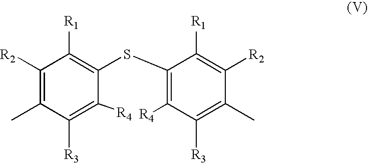 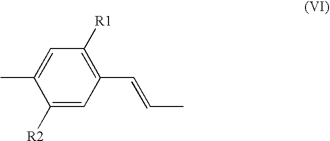
indoles of formula (VII) below: 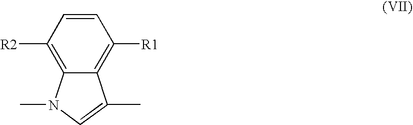
aromatic amides of formulae (VIIIa), (VIIIb), (VIIIc) and (VIIId) below: 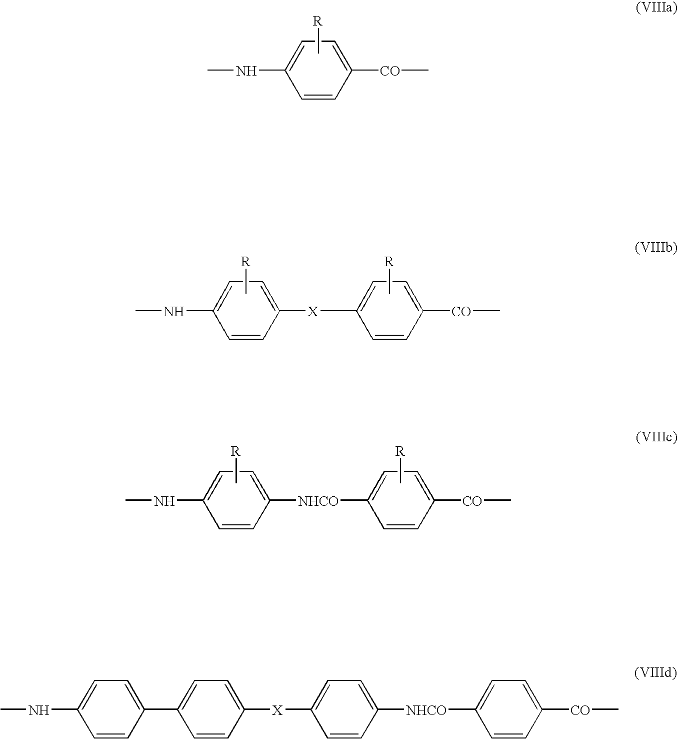
aromatic hydrazides of formulae (IXa), (IXb) and (IXc) below: 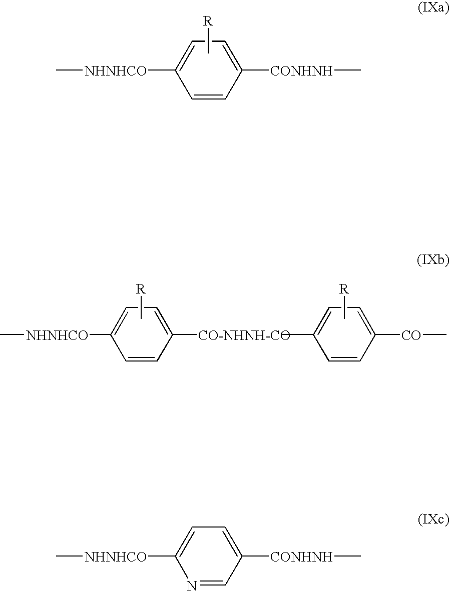
aromatic azomethines of formulae (Xa), (Xb) and (Xc) below: 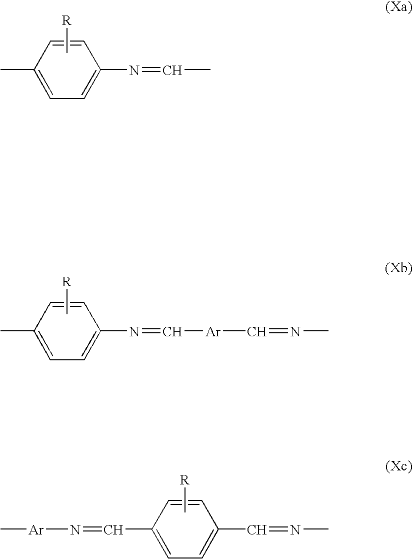
and aromatic esters of formulae (XIa), (XIb) and (Xlc) below: 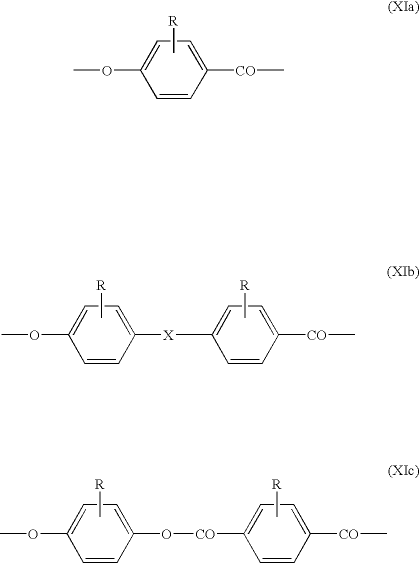
wherein in formulae (I) to (XI),
the radicals R, R₁, R₂, R₃, and R₄, which may be identical or different, are chosen from hydrogen, —R′ radicals, —OR′ radicals, —COOR′ radicals, and —OCOR′ radicals, wherein R′ is chosen from linear and branched C₁-C₂₀ alkyl radicals, halogen atoms, nitro radicals, cyano radicals, cyanoalkyl radicals, and solubilizing groups optionally comprising a spacer group that bonds to the ring;
Ar is a radical comprising a monoaromatic or a polyaromatic radical;
X is a radical chosen from —NHCO—, —O—, —S—, —SO₂—, —N═N—, —C(CH₃)₂—, —CH₂—, —CH═CH—, and —CH═N— radicals;
Z is a radical chosen from —CH═CH— and —C≡C— radicals.

3. The composition according to claim 2, wherein the solubilizing groups are chosen from:
carboxylic (—COOH) radicals and carboxylate radicals (—COO—M⁺) wherein M is chosen from alkali metals; alkaline-earth metals; organic amines; alkanolamines; and amino acids,
sulfonic (—SO₃H) and sulfonate (—SO₃⁻M⁺) radicals, M having the same definition as above,
primary, secondary and tertiary amine radicals,
quaternary ammonium radicals
hydroxyl radicals, and
poly((C₂-C₃)alkylene oxide) radicals.

4. The composition according to claim 3, wherein the quaternary ammonium radicals are —N(R′)₃⁺Z⁻ radicals,
wherein Z is chosen from Br, Cl, and (C₁-C₄)alkyl-OSO₃ radicals; and
R′, which may be identical or different, is chosen from linear and branched C₁ to C₂₀ alkyls, or two R's form a heterocycle with the nitrogen.

5. The composition according to claim 2, wherein the solubilizing groups are connected to the ring via a spacer group.

6. The composition according to claim 5, wherein the spacer group is a radical chosen from —R″—, —OR″—, —OCOR″— and —COOR″— radicals, wherein R″ is chosen from linear and branched C₁-C₂₀ alkyl radicals.

7. The composition according to claim 6, wherein the linear and branched C₁-C₂₀ alkyl radicals comprise at least one hetero atom.

8. The composition according to claim 2, wherein the radicals R, R₁, R₂, R₃, and R₄, which may be identical or different, are each chosen from hydrogen, and R′, —OR′, —OCOR′, and —COOR′ radicals, wherein R′ is chosen from linear and branched C₁-C₆ alkyl radicals, and from the following neutralized and non-neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, and —[(CH₂)₂O]ₓCH₂CH₂OCH₃ wherein x is an average number ranging from 0 to 200.

9. The composition according to claim 2, wherein at least one radical chosen from R₁, R₂, R₃, and R₄ is a solubilizing group.

10. The composition according to claim 1, wherein the at least one conductive polymer comprises at least one solubilizing group per repeating unit.

11. The composition according to claim 2, wherein the solubilizing groups are chosen from carboxylic acid groups; sulfonic acid groups; tertiary amine radicals; quaternary ammonium radicals; and also the salts thereof; wherein the carboxylic or sulfonic acid functions are optionally neutralized.

12. The composition according to claim 11, wherein the solubilizing groups are connected to the ring via a spacer group.

13. The composition according to claim 12, wherein the spacer group is chosen from C₁-C₂₀ alkyl radicals.

14. The composition according to claim 11, wherein the quaternary ammonium radicals are —N(R′)₃⁺Z⁻ radicals,
wherein Z is chosen from Br, Cl, and (C₁-C₄)alkyl-OSO₃ radicals and
R′, which may be identical or different, is chosen from linear and branched C₁-C₂₀ alkyl radicals.

15. The composition according to claim 2, wherein the at least one conductive polymer is chosen from polymers comprising at least one repeating unit of formula (IIIa), (IIIb) or (IIIc), wherein at least one radical chosen from R₁, R₂, R₃, and R₄ of formula (IIIa) or R₁ and R₂ of formulae (IIIb) or (IIIc) is a carboxylic acid solubilizing group, in neutralized or non-neutralized form, optionally connected to the ring via a spacer group, and wherein the other radicals are hydrogen atoms.

16. The composition according to claim 15, wherein the spacer group is chosen from linear and branched C₁-C₂₀ alkyl radicals.

17. The composition according to claim 1, wherein the at least one conductive polymer is present in an amount of at least 0.001% by weight relative to the total weight of the composition.

18. The composition according to claim 1, wherein the at least one conductive polymer is present in an amount of up to 50% by weight relative to the total weight of the composition.

19. The composition according to claim 1, wherein the at least one conductive polymer is present in an amount ranging from 0.1% to 50% by weight relative to the total weight of the composition.

20. The composition according to claim 1, wherein the at least one conductive polymer has a conductivity ranging from 1×0-5 to 5×10⁵ siemens/cm.

21. The composition according to claim 1, wherein the at least one rigid non-film-forming particle is chosen from mineral particles chosen from oxides and silicates,

22. The composition according to claim 21, wherein the oxides and silicates are chosen from those of alkali metals; alkaline-earth metals; silica; titanium dioxide; alumina; aluminosilicates; silicon carbide; silicon nitride; native metals; and alloys thereof.

23. The composition according to claim 22, wherein the native metals are chosen from silver and gold.

24. The composition according to claim 1, wherein the at least one rigid non-film-forming particle is chosen from polymer particles.

25. The composition according to claim 24, wherein the polymer particles have a glass transition temperature (Tg) greater than 50° C.

26. The composition according to claim 25, wherein the polymer particles have a glass transition temperature (Tg) greater than 70° C.

27. The composition according to claim 24, wherein the polymer constituting the at least one rigid non-film-forming particle is crosslinked.

28. The composition according to claim 24, wherein the polymer constituting the at least one rigid non-film-forming particle is an anionic polymer comprising ionized or ionizable anionic groups, which may be partially or totally neutralized.

29. The composition according to claim 24, wherein the polymer constituting the at least one rigid non-film-forming particle is a polymer or copolymer obtained by polymerization or copolymerization of a monomer or a blend of monomers chosen from acrylates and methacrylates of a linear, cyclic or branched C₁-C₁₀ alkyl; styrene, vinyltoluene, vinyl chloride, vinyl benzoate, vinyl tert-butylbenzoate, acrylic acid and methacrylic acid.

30. The composition according to claim 29, wherein the at least one rigid non-film-forming particle is chosen from particles of cationic latex.

31. The composition according to claim 1, wherein the at least one rigid non-film-forming particle is chosen from particles formed from conductive polymers.

32. The composition according to claim 1, wherein the cosmetically acceptable aqueous medium comprises water or a mixture of water and of at least one cosmetically acceptable solvent that is compatible with the rigid non-film-forming particles.

33. The composition according to claim 32, wherein the at least one cosmetically acceptable solvent is chosen from C₂-C₄ aliphatic monoalcohols, aromatic alcohols, glycols and glycol ethers, and polyols.

34. The composition according to claim 1, further comprising at least one non-fluorescent direct dye chosen from nonionic, cationic and anionic direct dyes.

35. The composition according to claim 34, wherein the at least one direct dye is chosen from nitrobenzene dyes, azo dyes, anthraquinone, naphthoquinone and benzoquinone dyes, indigoid dyes, triarylmethane-based dyes and natural dyes, and mixtures thereof.

36. The composition according to claim 34, wherein the direct dye is present in an amount ranging from 0.0005% to 12% by weight relative to the weight of the dye composition.

37. The composition according to claim 1, further comprising at least one fluorescent dye.

38. The composition according to claim 37, wherein the at least one fluorescent dye is chosen from substituted 4-aminophenylethenylpyridinium derivatives; naphthalimides; cationic and non-cationic coumarins; xanthenodiquinolizines; azaxanthenes; naphtholactams; azlactones; oxazines; thiazines; dioxazines, pyrenes and nitrobenzoxadiazoles, and mixtures thereof.

39. The composition according to claim 1, further comprising at least one optical brightener.

40. The composition according to claim 39, wherein the at least one optical brightener is chosen from stilbene derivatives, coumarin derivatives, oxazole and benzoxazole derivatives, and imidazole derivatives.

41. The composition according to claim 37, further comprising at least one optical brightener.

42. The composition according to claim 41, wherein the at least one optical brightener is chosen from stilbene derivatives, coumarin derivatives, oxazole and benzoxazole derivatives, and imidazole derivatives.

43. The composition according to claim 41, wherein the at least one fluorescent dye and the at least one optical brightener are each present in an amount ranging from 0.01% to 20% by weight relative to the total weight of the composition.

44. The composition according to claim 43, wherein the at least one fluorescent dye and the at least one optical brightener are each present in an amount ranging from 0.05% to 10% by weight relative to the total weight of the composition.

45. The composition according to claim 1, wherein the composition is in the form of a lotion, a spray, a mousse, a cream or a gel.

46. The composition according to claim 1, wherein the composition is packaged under pressure in a pump-dispenser bottle or in a pressurized aerosol device.

47. The composition according to claim 46, the composition further comprising at least one propellant.

48. The composition according to claim 47, wherein the at least one propellant is chosen from volatile hydrocarbons, chloro hydrocarbons and fluoro hydrocarbons and mixtures thereof; carbon dioxide, nitrous oxide, dimethyl ether, nitrogen and compressed air.

49. The composition according to claim 47, wherein the at least one propellant is present in an amount ranging from 5% to 90% by weight relative to the total weight of the composition in the aerosol device.

50. A process for treating keratin fibers comprising:
applying a composition to wet or dry fibers, the composition comprising, in a cosmetically acceptable medium:
(a) at least one rigid non-film-forming particle, and
(b) at least one conductive polymer, and then drying or leaving to dry the fibers thus treated.

51. The process according to claim 50, wherein the keratin fibers are human keratin fibers.

52. The process according to claim 51, wherein the human keratin fibers are hair.

53. The process according to claim 50, wherein the at least one conductive polymer comprises at least one repeating unit chosen from the following formulae:
anilines of structure (I) below: 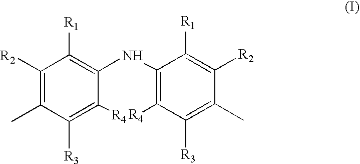
pyrroles of structures (IIa) and (IIb) below: 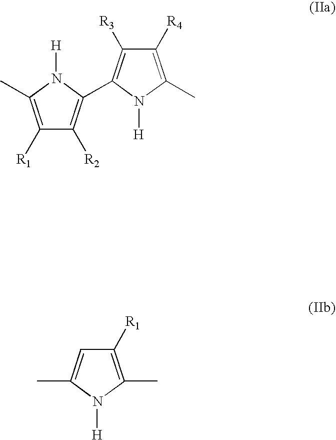
thiophenes or bisthiophenes of formulae (IIIa), (IIIb) and (IIIc) below: 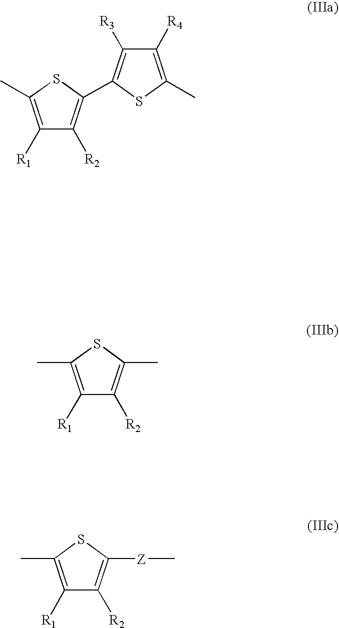
furans of formula (IV) below: 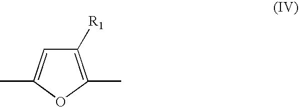
para-phenylene sulfildes of structure (V) below: 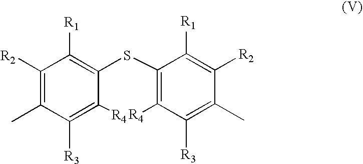
para-phenylenevinylenes of formula (VI) below: 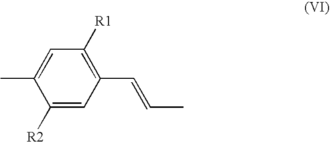
indoles of formula (VII) below: 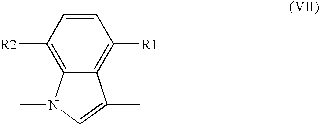
aromatic amides of formulae (VIIIa), (VIIIb), (VIIIc) and (VIIId) below: 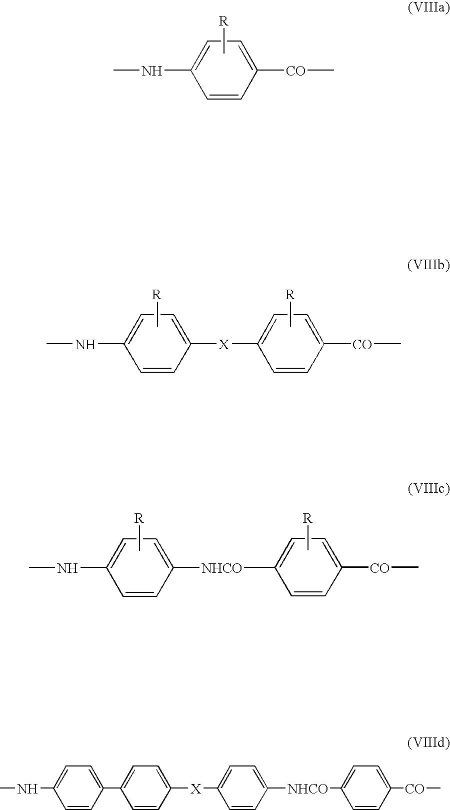
aromatic hydrazides of formulae (IXa), (IXb) and (IXc) below: 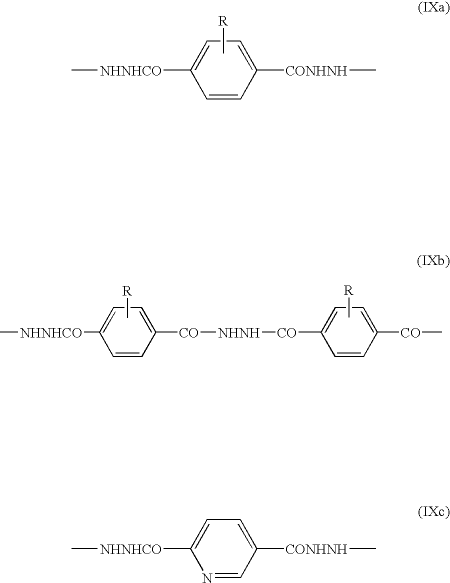
aromatic azomethines of formulae (Xa), (Xb) and (Xc) below: 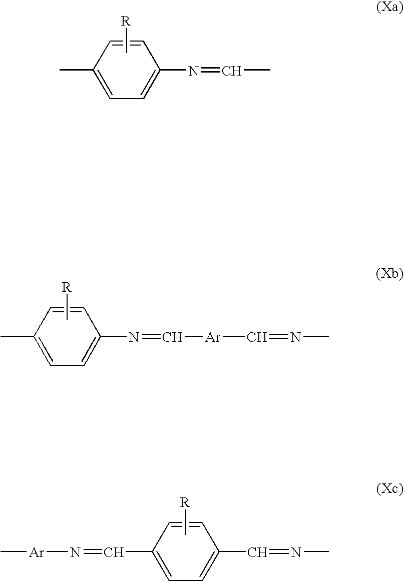
and aromatic esters of formulae (XIa), (XIb) and (XIc) below: 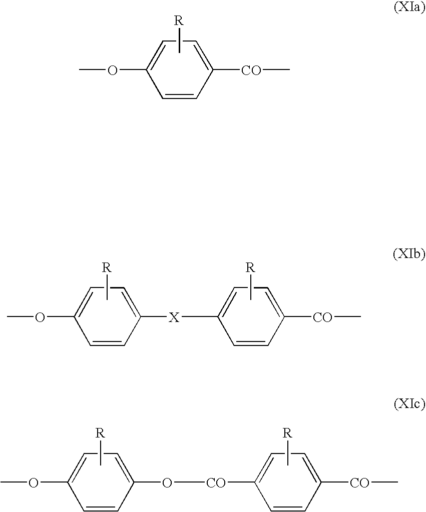
wherein in formulae (I) to (XI),
the radicals R, R₁, R₂, R₃, and R₄, which may be identical or different, are chosen from hydrogen, —R′ radicals, —OR′ radicals, —COOR′ radicals, and —OCOR′ radicals, wherein R′ is chosen from linear and branched C₁-C₂₀ alkyl radicals, halogen atoms, nitro radicals, cyano radicals, cyanoalkyl radicals, and solubilizing groups optionally comprising a spacer group that bonds to the ring;
Ar is a radical comprising a monoaromatic or a polyaromatic radical;
X is a radical chosen from —NHCO—, —O—, —S—, —SO₂—, —N═N—, —C(CH₃)₂—, —CH₂—, —CH═CH—, and —CH═N— radicals;
Z is a radical chosen from —CH═CH— and —C—C— radicals.

54. The composition according to claim 53, wherein the solubilizing groups are chosen from:
carboxylic (—COOH) radicals and carboxylate radicals (—COO—M⁺) wherein M is chosen from alkali metals; alkaline-earth metals; organic amines; alkanolamines; and amino acids,
sulfonic (—SO₃H) and sulfonate (—SO₃⁻M⁺) radicals, M having the same definition as above,
primary, secondary and tertiary amine radicals,
quaternary ammonium radicals
hydroxyl radicals, and
poly((C₂-C₃)alkylene oxide) radicals.

55. The composition according to claim 54, wherein the quaternary ammonium radicals are —N(R′)₃⁺Z radicals,
wherein Z is chosen from Br, Cl, and (C₁-C₄)alkyl-OSO₃ radicals; and
R′, which may be identical or different, is chosen from linear and branched C₁ to C₂₀ alkyls, or two R's form a heterocycle with the nitrogen.

56. A process for shaping and/or holding the hairstyle comprising applying a hair product base to the hair, the hair product base comprising a composition comprising, in a cosmetically acceptable medium:
(a) at least one rigid non-film-forming particle, and
(b) at least one conductive polymer.

57. A process for imparting to keratin fibers an optical effect, comprising applying to the keratin fibers a composition comprising, in a cosmetically acceptable medium:
(a) at least one rigid non-film-forming particle, and
(b) at least one conductive polymer.

58. The process according to claim 57, wherein the optical effect is providing sheen to the keratin fibers.
